(19) United States
(12) Patent Application Publication
(10) Pub. No.: US 2004/0034320 A1
(43) Pub. Date: Feb. 19, 2004

(54) Anti-reflux feeding tube

(76) Inventor: Daniel Rogers Burnett, Menlo Park, CA (US)

Correspondence Address: Colin R. Crossman, 3204 Cabarrus Dr., Greensboro, NC, 27407, US

(21) Appl. No.: 10/636,804

(22) Filed: Aug. 8, 2003

Related U.S. Application Data
(60) Provisional application No. 60/403,050, filed on Aug. 13, 2002.
(60) Provisional application No. 60/434,531, filed on Dec. 19, 2002.

(51) Int. Cl. A61M 29/00; A61M 37/00; A61M 25/00

(52) U.S. Cl. 604/96.01; 604/523; 604/103.08

(57) ABSTRACT

A catheter for delivering fluids to a patient, while preventing reflux of the tube feeds and the conditions associated therewith. The catheter comprises a tube, a barrier to prevent reflux, and a means for stomach evacuation. The catheter has several configurations, depending on the specific requirements of the patient's condition. Also provided are safety measures for the patient's protection.

### RELATED APPLICATION

This application claims the priority of U.S. Provisional Application Serial No. 60/403,050, filed on Aug. 13, 2002 and Provisional Application Serial No. 60/434,531 filed on Dec. 19, 2002. The aforementioned provisional applications are incorporated by reference herein for all purposes.

### FIELD OF THE INVENTION

The present invention pertains to an enteral feeding tube, specifically an enteral feeding tube designed to prevent reflux of the feeding contents.

### BACKGROUND OF THE INVENTION

In medicine there are a variety of chronic and acute conditions resulting in the need to nutritionally supplement a patient. Typically, if a patient is going to be without food for greater than one week, then they will require some sort of nutritional supplementation.

Currently, the only two options for nutritional supplementation involve feeding the patient through their gastrointestinal tract or through an intravenous route. The gastrointestinal route is associated with superior immunity and fewer complications, and, as such, is used more frequently.

Enteral tube feeding is delivered to an estimated 900,000 patients in the U.S. each year for conditions including ventilator dependence, trauma, and various forms of dysphagia. While usually effective in the restoration of nutritional status of the patient, enteral tube feeding has been associated with serious complications. One of the most serious and, unfortunately, one of the most common problems with enteral tube feeding is the development of aspiration pneumonia. Studies have shown that up to 50% of patients on chronic enteral feeds develop findings consistent with aspiration. Despite the alarming rate of this potentially fatal complication, existing devices for the delivery of enteral tube feeds have failed to adequately address or solve this problem.

Several recent studies have established that duodenogastric reflux, gastric colonization with intestinal bacteria, and gastroesophageal regurgitation may be the primary factors leading to the development of pulmonary aspiration and the morbidity attributable to that condition.

### SUMMARY OF THE INVENTION

The present invention has grown out of a body of existing research demonstrating that tube feed reflux persists regardless of the site of tube feed delivery. The present invention addresses the need for prevention of tube feed reflux through the use of a gastric evacuator and the incorporation of a rigid or temporarily inflatable barrier. When inflated, this barrier prevents the retrograde flow of the delivered tube feeds to the stomach and the lungs. The inflatable barrier is easily positioned during the tube feeding and deflated once the intestine has been emptied of the delivered nutrients. To ensure that the pressures generated in the intestine remain below the threshold at which mucosal damage occurs, multiple safety mechanisms are preferably incorporated into either the inflatable barrier, or the feeding tube, or both. The gastric evacuator component preferably decompresses the stomach to prevent reflux of gastric secretions.

### BRIEF DESCRIPTION OF THE DRAWINGS

FIG. 1—A cross-sectional view of the device being inserted percutaneously.

FIGS. 2A-C—Cross-sectional views of the Anti-Reflux Bulb (ARB) of the present invention in three stages of expansion and inflation.

FIG. 3—A cross-sectional view of the device after gastric insertion, but prior to release of the vacuum seal on the ARB.

FIG. 4—A cross-sectional view of the device after gastric insertion, with partial release of the vacuum seal on the ARB to encourage transpyloric migration of the tip of the device.

FIGS. 5A-C—A cross-sectional view of the function of the inserted device, including: A) Transpyloric passage, B) Full expansion of the foam in the distal balloon, and D) Inflation of the ARB across the pyloric valve.

FIGS. 6A-B—A cross-sectional view of the two different mechanisms of insertion of the device: A) Nasal insertion and B) Percutaneous Insertion.

FIGS. 7A-C—A cross-sectional view of three possible alternative embodiments of the device including A) An hourglass-shaped balloon that requires retraction against the pyloric valve for proper placement, B) A permanent flanged tube structure designed to be anchored in place across the pyloric valve with sutures, staples and C) The device of FIG. 2 in which a proximal, gastrically-retained balloon is utilized in order to ensure proper placement of the inflating ARB.

### DETAILED DESCRIPTION OF THE PREFERRED EMBODIMENTS

The Anti-Reflux Feeding Tube design of the present invention consists of a triple-lumen tube, manufactured from any biocompatible material, which preferably interfaces with a programmable pump. The various components of the Anti-Reflux Feeding Tube System are as follows.

The first lumen serves as the route of administration of tube feeds to the patient. The first lumen optionally, but preferably, incorporates some of the safety mechanisms below.

The second lumen will terminate in a perforated sleeve in the stomach. Optionally, the perforated sleeve may be substituted with a mesh, or another suitable method for preventing solids from clogging the tube, or left off entirely. When connected to suction, the perforated sleeve provides effective evacuation of gastric secretions during the delivery of the tube feeds.

The third lumen terminates in an inflatable balloon just prior to the opening of the larger lumen. This lumen, which will be the port of entry for the air required to inflate and deflate the anti-reflux balloon, may optionally slide freely within the optional Anti-Reflux Feeding Tube sheath allowing for the extension and retraction of the attached distal anti-reflux balloon (ARB).

The ARB envisioned by the inventor entails a long thin balloon which when inflated is larger than the diameter of the relaxed pyloric sphincter, but which is still smaller than the intestinal lumen. The balloon diameter will be between 0.5 mm and 75 mm, preferably between 5 mm and 15 mm. An additional, optional, embodiment of the balloon includes ridges, ribs, or bumps along the surface of the balloon to increase the balloon's ability to form an effective, stationary, seal with the pyloric valve.

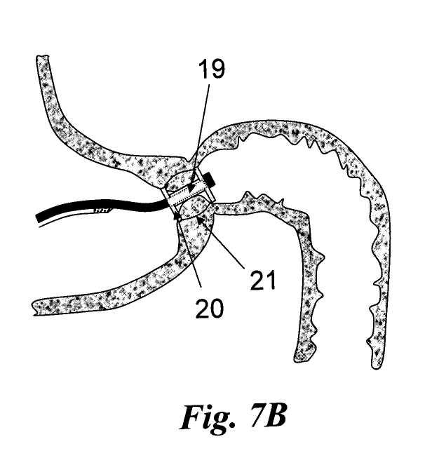

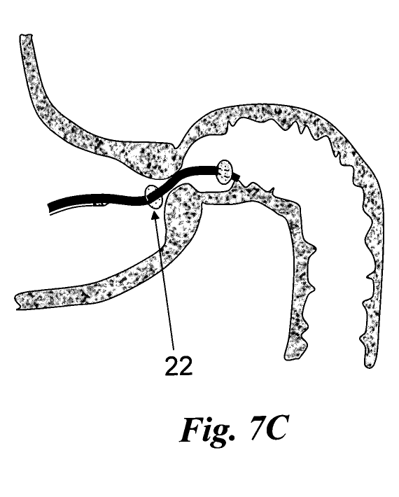

The ARB contains a self-expanding mass of foam in the distal portion of its preferably long cylinder shaped balloon which, when connected to a vacuum and sealed, is compressed facilitating insertion into the stomach and then duodenum. Once the duodenal position of the ARB is confirmed, the vacuum can be released allowing the foam to expand to a size large enough to prevent return of the fully expanded tip back through the pyloric valve. Even fully expanded, the volume of the foam within the balloon is preferably small enough to not interfere with the flow of solid contents through the duodenum. The foam in the balloon can also be partially expanded in order to encourage transpyloric passage of the tip of the device from the stomach to the duodenum. Once in the duodenum, it can then be fully expanded to retain its position beyond the pyloric valve. In this conformation, the fully inflated ARB is long enough to interface with the pyloric valve as long as the foam-filled tip of the ARB is in the duodenum (see FIG. 7A). Additionally, the proximal portion of the device residing in the stomach may incorporate a larger positional balloon, as well, which is large enough to prevent pyloric passage (see FIG. 7C), but small enough to allow comfortable retention in the gastric space. This feature will allow the inflatable portion of the ARB to be accurately located in the pyloric sphincter at all times.

In a second manifestation, the ARB straddles the pyloric valve when inflated. In this embodiment, the ARB is hourglass shaped with a distal and a proximal bulb which can be inflated across the sphincter. Thus, once it is confirmed that the foam-filled tip is in the duodenum and the ARB is retracted against the pyloric sphincter and inflated, the pyloric sphincter is successfully occluded.

A third modality envisioned by the inventor involves an endoscopically placed flanged tube design in which the two flanges of the tube are designed to be placed on either side of the pyloric valve then sutured or stapled together. Thus, this design provides a relatively permanent mechanism for preventing duodenogatsric reflux, even with tube feeds. Preferably there will be an opening or flap valve that will allow drainage of gastric secretions which will be closed with influx of tube feeds into the tube. This configuration may remain in the pyloric valve position indefinitely and would be useful in patients requiring relatively long-term nutritional support.

The preferred application of the device entails placement of the feeding tube either nasally or percutaneously with the feeding tube outlet in the intestine. After placement in the duodenum, the vacuum seal on the ARB will be fully removed and the self-expanding foam inside the distal portion of the balloon will increase in size until it can no longer pass the pyloric valve.

The tube feed delivery rate can vary from bolus feeding to nearly continuous feeding. It is expected, though, that for patients with healthy gastrointestinal tracts the tube feed will be delivered as a bolus due to the fact that this is the most physiologic and convenient delivery format for the patient and their caregivers.

The Anti-Reflux Feeding Tube design also optionally, but preferably, contains two important safety features to ensure that undue pressure is not applied to the intestinal wall. The first optional, but preferable, safeguard involves regulation of the inflation pressure of the occlusive balloon through the use of small balloon with increased compliance at the proximal end of the tube. This small, compliant safety balloon, placed externally and in-line with the air-containing lumen through which the occlusive balloon is inflated, will begin to expand once an adequate occlusive pressure of about 15-50 mmHg has been reached. This pressure is considered safe with respect to intestinal mucosa and musculature, even with prolonged exposure. If, for some reason, inflation continues beyond the adequate pressure level, the safety balloon is also designed to rupture prior to reaching a critical pressure of about 60-100 mmHg, which is the maximum average pressure associated with physiologic peristalsis. The second optional, but preferable, safeguard is a similar area of increased compliance at the proximal end of the tube feed lumen. If the pressure in the tube feed lumen increases beyond 15-50 mmHg, this area of increased compliance will begin to warn the healthcare provider by expanding and, if the pressure reaches 60-100 mmHg, rupturing. Thus, as with the inflation lumen, the pressure will be relieved prior to damaging the intestinal mucosa. Used together, these safety features provide a redundant safeguard against excess intraluminal pressures.

### DESCRIPTION OF THE DRAWINGS

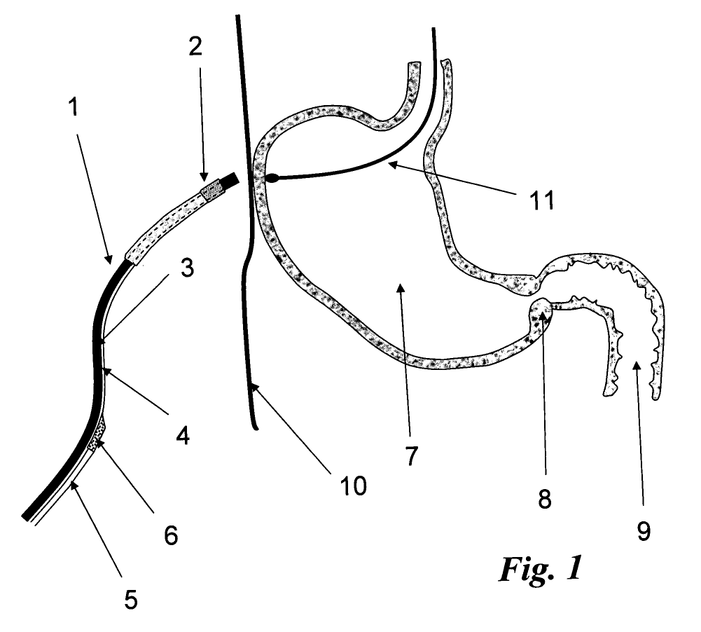

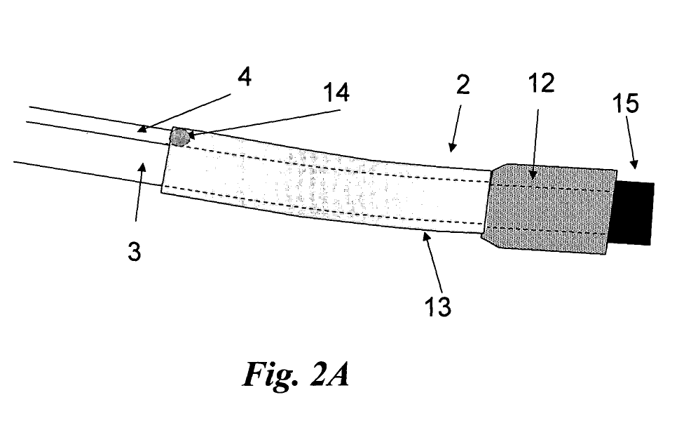

As can be seen in FIG. 1, the present invention provides a feeding tube 1 which can be advanced into the stomach 7 either nasally, or, as illustrated in FIG. 1, percutaneously. The tube has multiple elements including: an Anti-Reflux Bulb (ARB) 2, a feeding tube lumen 3, an inflation lumen 4, a lumen provided for evacuation of the gastric contents 5, and a perforated tip 6 on the end of 5. When inserted percutaneously, the tube 1, is inserted through the skin 10 into the stomach 7 through the use of a guide wire 11. After placement in the stomach 7, the tube 1 will then be advanced beyond the pyloric valve 8 into the duodenum 9.

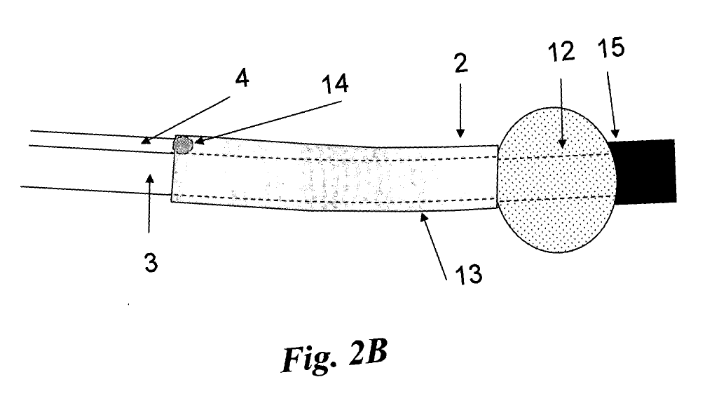

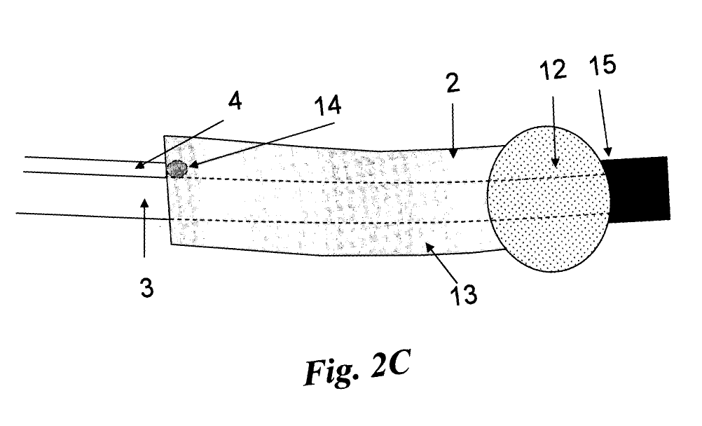

FIG. 2 shows three conformations of the preferred embodiment of the invention. First, the ARB 2 is configured to have two bulbs on its terminal tip, a distal bulb 12, and a long proximal bulb 13. Once the ARB 2 is positioned across the pyloric valve 8, bulb 12 would reside in the duodenum, while bulb 13 would span the pyloric valve 8. The gastric portion of the balloon 13 also has an aperture 14 to allow air to move in and out of the ARB 2. The feeding tube lumen 3 is continuous through the center of the ARB 2 and terminates in a soft tip 15, preferably designed to not irritate the bowel wall. The intestinal bulb 12 of the balloon also preferably incorporates expanding foam within the balloon. This foam, when allowed to expand as shown in FIG. 2B, results in retention of the feeding tube outlet in the duodenum 9 due to the fact that the foam preferably expands to a volume that does not permit it to reflux past the pyloric valve 8. The foam, though, preferably does not expand to a size large enough to interfere with the flow of duodenal contents. The fully inflated ARB, FIG. 2C, is shown with its two bulbs fully expanded, so as to span the pyloric valve 8 when positioned correctly.

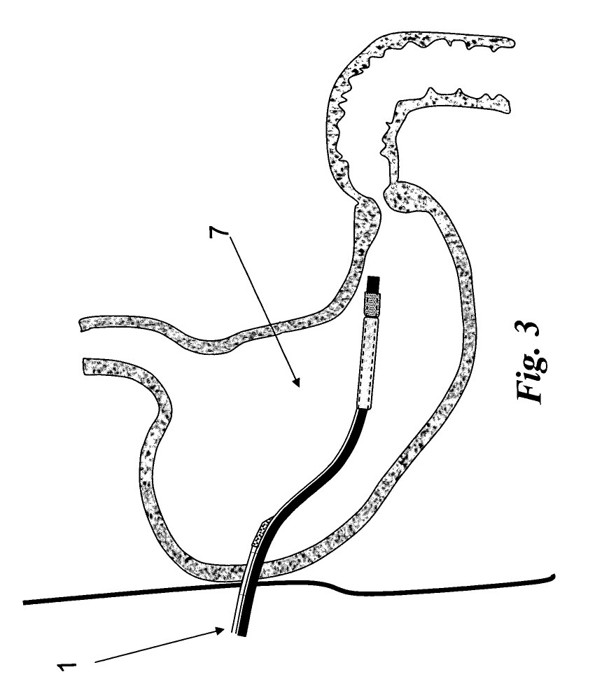

As can be seen in FIG. 3, the feeding tube 1 is advanced into the stomach 7 before passage into the duodenum.

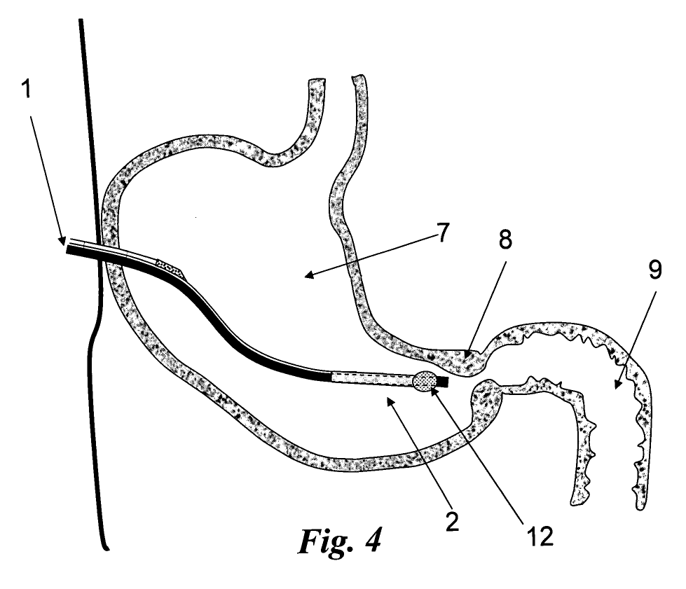

FIG. 4 illustrates the removal of vacuum on the ARB 2 and the partial expansion of the foam balloon 12 to promote transpyloric passage int the duodenum 9.

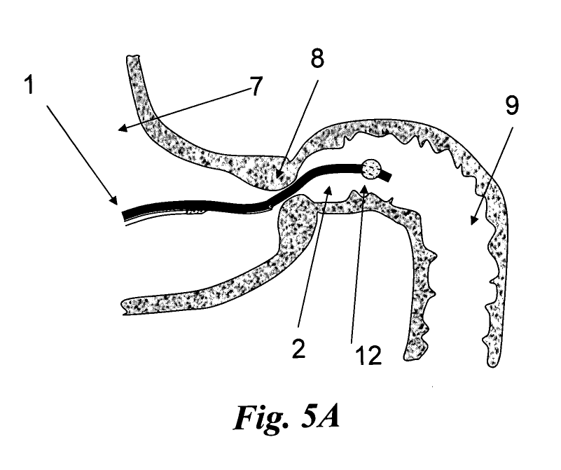

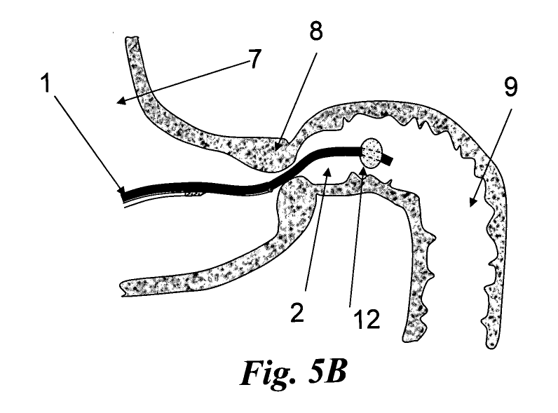

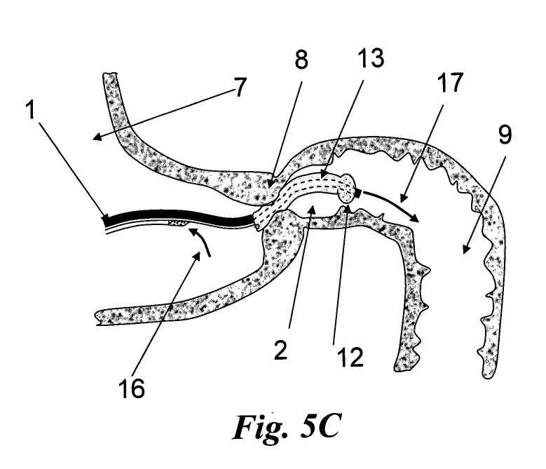

FIGS. 5A-C illustrate the transpyloric passage of the ARB 2 and its positioning. As can be seen in FIG. 5B, once the ARB 2 is in place in the duodenum 9, the foam-filled tip 12 is allowed to fully expand trapping the ARB in the duodenum. In FIG. 5C, the ARB 2 is retracted to the pyloric valve 8 at which point it is inflated, spanning the pyloric valve and tube feed and gastric evacuation can commence according to the programmed protocol of the complementary pump. In addition, FIG. 5C shows the gastric fluid being drained from the stomach 16, and the tube feeds being deposited 17 into the duodenum 9.

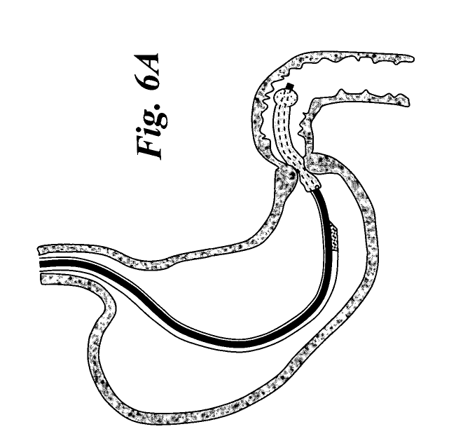

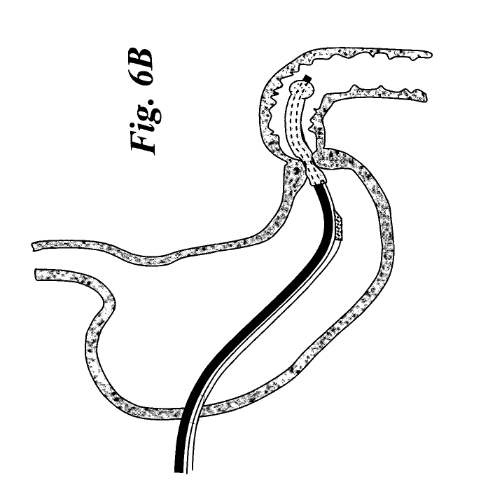

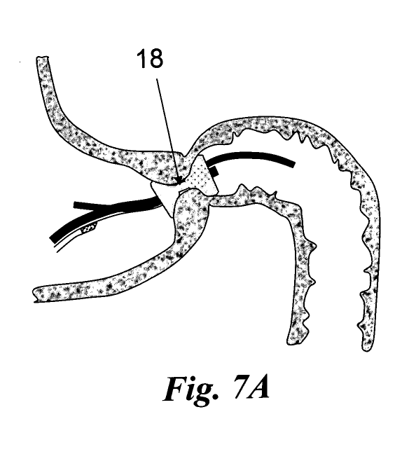

As can be seen in FIG. 6, the present invention may be inserted nasally FIG. 6A or percutaneously FIG. 6B.

Lastly, additional embodiments contemplated by the inventor are illustrated in FIGS. 7A-C. In FIG. 7A, an alternative embodiment, which employs a hourglass-shaped balloon 18 spanning the pyloric valve 8 is shown. The advantage of this embodiment is the snug fit on the pyloric valve while the disadvantage is the requirement for precise placement not necessary in the preferred embodiment. The embodiment illustrated FIG. 7B shows a relatively permanent tube 19, fitted securely across the pyloric valve by flanges 20 that are optionally sutured, stapled or affixed 21 in place. This embodiment would remain in place and optionally incorporates a valve to prevent reflux of duodenal contents, but which will allow drainage of gastric secretions into the duodenum. In FIG. 7C, the preferred embodiment is equipped with an additional proximal foam-filled balloon 22 which has been designed to be retained in the gastric space. This will allow the pyloric-occlusion balloon to be snugly held across the pyloric valve while not requiring permanent fixation and occlusion of the pylorus as in FIG. 7B.

Not shown, but also envisioned by the inventor are the embodiments employing an inner and outer catheter for the feeding tube 1, which, when agitated will break deposits free from the inner lumen. Also envisioned but not illustrated is the catheter valve component which may consist of a mechanism such as a flap of flexible material or ball valve over the terminal end 15 of the feeding tube 1 or even an active valve opened and closed through EMF or radio wave communication.

While these are the preferred embodiments in which the pyloric valve is occluded to encourage antegrade flow of gastrointestinal contents, the device could employ any mechanism that provides unidirectional flow of enteral feeding contents within the gastrointestinal tract.

As considered above, this could involve an electronic valve triggered via communication across the tissues of the human body through EMFs, or other means of communication, allowing opening of the aperture 15 only at selected times. The valve of the device can take many shapes and the device can be manufactured from any of a variety of materials with the only requirement being that of biocompatibility.

1. A catheter for enteral feeding, comprising:
a tube, having a proximal end and a distal end; and
a barrier means for preventing tube feed reflux across the pyloric valve of the stomach, wherein said means for preventing tube feed reflux is located proximal to the end of said tube.

2. A catheter as recited in claim 1, further comprising an evacuation means for evacuating stomach contents during feeding, wherein said means for evacuating stomach contents is located proximal to the means for preventing tube feed reflux.

3. A catheter as recited in claim 2, wherein said evacuation means is a tube for draining stomach fluids.

4. A catheter as recited in claim 2, wherein said evacuation means is a lumen for draining stomach fluids.

5. A catheter as recited in claim 2, wherein said evacuation means includes a filter means.

6. A catheter as recited in claim 6, wherein said filter means is a mesh.

7. A catheter as recited in claim 6, wherein said filter means is a perforated tube.

8. A catheter as recited in claim 6, wherein said filter means is a porous material.

9. A catheter as recited in claim 1, wherein said barrier means is an inflatable balloon.

10. A catheter as recited in claim 9, further comprising a compressible foam component, whereby said compressible foam component is contained within said inflatable membrane.

11. A catheter as recited in claim 9, wherein said balloon has a textured surface.

12. A catheter as recited in claim 9, wherein said balloon is ribbed.

13. A catheter as recited in claim 1, wherein said barrier means is a pair of flanges.

14. A catheter as recited in claim 13, wherein said barrier means includes a valve.

15. A catheter as recited in claim 9, wherein said balloon is spheroid in shape.

16. A catheter as recited in claim 9, wherein said balloon has an hourglass shape.

17. A catheter as recited in claim 9, wherein said balloon is elongated coaxially with the tube.

18. A catheter as recited in claim 17, wherein the distal end of said balloon has a longer cross-section than the proximal end of said balloon.

19. A catheter as recited in claim 1, wherein said evacuation means includes a sleeve.

20. A catheter as recited in claim 9, further comprising an inflation safeguard means for regulating the inflation of said balloon.

21. A catheter as recited in claim 20, wherein said inflation safeguard means is a second balloon, whereby said second balloon is sufficiently compliant to rupture at an internal pressure of about 100 mmHg.

22. A catheter as recited in claim 1, whereby said tube includes a region of increased compliance distal to the proximal end of the tube, said region sufficiently compliant to rupture at an internal pressure of 50-100 mmHg.

23. A catheter as recited in claim 1, further comprising an outer sheath partially encompassing said tube.

24. A catheter as recited in claim 9, further comprising a means to restrict additional catheter penetration beyond the pyloric valve.

25. A catheter as recited in claim 24, wherein said means to restrict additional catheter penetration is an enlarged region of the inflatable balloon, located at the proximal end of the inflatable balloon.

26. A catheter as recited in claim 24, wherein said means to restrict additional catheter penetration is a secondary balloon, located proximal to the inflatable balloon.

27. A catheter as recited in claim 24, wherein said means to restrict additional catheter penetration is a soft plastic element, located proximal to the inflatable balloon.

28. A catheter as recited in claim 24, wherein said means to restrict additional catheter penetration is a foam element, located proximal to the inflatable balloon.

29. A catheter as recited in claim 25, further comprising a compressible foam component, whereby said compressible foam component is contained completely within said inflatable balloon.

30. A catheter as recited in claim 26, further comprising a compressible foam component, whereby said compressible foam component is contained completely within said secondary balloon.

31. A catheter for enteral feeding, comprising:
a tube, having a proximal end, a distal end, and first lumen, wherein said first lumen is in fluid communication with the external environment near the distal end of the tube; and
a first barrier, proximal to the distal end of the tube, whereby said barrier prevents reflux across the pyloric valve of the stomach.

32. A catheter as recited in claim 31, further comprising a second lumen, whereby the second lumen is in fluid communication with the external environment at a point proximal to the distal end of the tube.

33. A catheter as recited in claim 32, further comprising a filter in line with the second lumen, whereby the filter prevents solids from the stomach cavity from clogging the second lumen.

34. A catheter as recited in claim 33, wherein said filter is a mesh.

35. A catheter as recited in claim 33, wherein said filter is a porous material.

36. A catheter as recited in claim 33, wherein said filter is a perforation.

37. A catheter as recited in claim 31, wherein said barrier is a pair of flanges.

38. A catheter as recited in claim 32, further comprising a valve in the barrier.

39. A catheter as recited in claim 31, wherein said tube further comprises a third lumen, and wherein said barrier is an inflatable membrane in fluid communication with said third lumen.

40. A catheter as recited in claim 39, wherein said inflatable membrane is extended in the direction coaxial to the catheter, and has a first enlarged region near the distal end of the catheter, and a non-enlarged region extending toward the proximal end of the catheter, whereby such enlarged region prevents tube return through the pyloric valve, and the non-enlarged region provides a site for the pyloric valve to interact with the catheter, and thereby prevents reflux of the tube feeds into the stomach and lungs.

41. A catheter as recited in claim 39, wherein said inflatable membrane has a textured outer surface.

42. A catheter as recited in claim 39, wherein said inflatable membrane has a ribbed outer surface.

43. A catheter as recited in claim 39, further comprising a compressible foam component, whereby said compressible foam component is contained within said inflatable membrane.

44. A catheter as recited in claim 39, further comprising an occlusive balloon, the occlusive balloon connected inline with the third lumen, whereby the occlusive balloon is sufficiently compliant to rupture at an internal pressure of 60-100 mmHg.

45. A catheter as recited in claim 31, further comprising a safety balloon, the safety balloon connected inline with the first lumen, whereby the safety balloon is sufficiently compliant to rupture at an internal pressure of 60-100 mmHg.

46. A catheter as recited in claim 31, further comprising an exterior sheath, whereby said exterior sheath partially encompasses the tube.

47. A catheter as recited in claim 31, further comprising a second barrier to restrict additional catheter penetration beyond the pyloric valve, whereby said second barrier is located proximal to the first barrier.

48. A catheter as recited in claim 47, wherein said second barrier is a soft plastic element, located proximal to the inflatable balloon.

49. A catheter as recited in claim 47, wherein said second barrier is a foam element, located proximal to the inflatable balloon.

50. A catheter as recited in claim 39, wherein said inflatable membrane includes an second enlarged region, located at the proximal end of the inflatable membrane, whereby such second enlarged region restricts additional catheter penetration beyond the pyloric valve.

51. A catheter as recited in claim 39, further comprising a secondary inflatable membrane, located proximal to the inflatable balloon, whereby such secondary inflatable membrane restricts additional catheter penetration beyond the pyloric valve.

52. A catheter as recited in claim 50, further comprising a compressible foam component, whereby said compressible foam component is contained completely within said second enlarged region.

53. A catheter as recited in claim 51, further comprising a compressible foam component, whereby said compressible foam component is contained completely within said second inflatable membrane.

54. A catheter, comprising:
a first channel, having a proximal end and a distal end;
a compressible foam component, attached to the first channel proximal to the distal end of said first channel;
a reversibly inflatable membrane, having an interior and an exterior, attached to the first channel, forming a gas and fluid impermeable seal with the outer surface of said first channel, and encompassing the compressible foam component;
a second channel, transverse to the inflatable membrane, having a proximal end and a distal end, the distal end of which lies in the interior of said inflatable membrane and is in fluid communication therewith; and
a third channel, associated with said first channel, having a proximal end and a distal end, the distal end of the third channel terminating proximal to the inflatable membrane, whereby the third channel is in fluid communication with the external environment.

55. A catheter as recited in claim 54, wherein the first, second, and third channels are separate tubes.

56. A catheter as recited in claim 54, wherein the first, second, and third channels are separate lumens of a single tube.

57. A catheter as recited in claim 54, wherein the outer surface of said reversibly inflatable membrane is textured.

58. A catheter as recited in claim 54, wherein said reversibly inflatable membrane is ribbed.

59. A catheter as recited in claim 54, wherein said reversibly inflatable membrane elongated coaxially with the first channel, and has an enlarged region near the distal end of the first channel.

60. A catheter as recited in claim 59, further comprising a second enlarged region at the proximal end of the reversibly inflatable membrane, whereby such second enlarged region prevents additional progression of the catheter beyond the pyloric valve.

61. A catheter as recited in claim 54, further comprising a compressible foam component, whereby said compressible foam component is contained completely within said reversibly inflatable membrane.

62. A catheter as recited in claim 60, further comprising a compressible foam component, whereby said compressible foam component is contained completely within said second enlarged region.

63. A catheter as recited in claim 54, further comprising a second reversibly inflatable membrane, whereby said compressible foam component is contained completely within said reversibly inflatable membrane, located proximal to the first reversibly inflatable membrane, whereby such second reversibly inflatable membrane prevents additional progression of the catheter beyond the pyloric valve.

64. A catheter as recited in claim 54, further comprising an occlusive balloon, the occlusive balloon connected inline with the third channel, whereby the occlusive balloon is sufficiently compliant to rupture at an internal pressure of 60-100 mmHg.

65. A catheter as recited in claim 54, further comprising a safety balloon, the safety balloon connected inline with the first channel, whereby the safety balloon is sufficiently compliant to rupture at an internal pressure of 60-100 mmHg.

66. A catheter as recited in claim 54, further comprising an exterior sheath, whereby said exterior sheath partially encompasses the tube.

67. A catheter as recited in claim 54, further comprising a filter in line with the third channel, whereby the filter prevents solids from the stomach cavity from clogging the second lumen.

68. A catheter as recited in claim 67, wherein said filter is a mesh.

69. A catheter as recited in claim 67, wherein said filter is a porous material.

70. A catheter as recited in claim 67, wherein said filter is a perforation.
